(19) United States
(12) Patent Application Publication
(10) Pub. No.: US 2022/0047822 A1
(43) Pub. Date: Feb. 17, 2022

(54) Accessories for Inhalers

(71) Applicant: BREATHESUITE INC., St. John's, Newfoundland and Labrador (CA)

(72) Inventor: Brett Vokey, St. John's (CA)

(21) Appl. No.: 17/274,474

(22) PCT Filed: Sep. 27, 2019
(86) PCT No.: PCT/IB2019/058254

§ 371 Date: Mar. 9, 2021

Related U.S. Application Data
(60) Provisional application No. 62/737,427, filed on Sep. 27, 2018.

(51) Int. Cl. A61M 15/00; G16H 20/13

(52) U.S. Cl. CPC A61M 15/0068; A61M 15/0025; G16H 20/13; A61M 2205/332; A61M 2205/3327; A61M 2205/3334; A61M 2205/582; A61M 2205/581; A61M 2205/3375; A61M 2205/3553; A61M 2205/52

(57) ABSTRACT

An example accessory for an inhaler includes: a housing to couple to the inhaler; an accelerometer disposed in the housing, the accelerometer to obtain (i) motion data representing motion of the inhaler and (ii) orientation data representing an orientation of the inhaler; a microphone disposed in the housing, the microphone to obtain audio data for determining an inhalation rate of a user of the inhaler; a force sensor disposed in the housing, the force sensor to obtain force data representing force applied to the accessory; a communications interface disposed in the housing; a memory disposed in the housing; and a processor disposed in the housing, the processor interconnected with the communications interface and the memory the processor to: detect actuation of the inhaler based on the force data applied to the accessory; and generate dosage administration data based on the motion data, the orientation data and the audio data.

### CROSS-REFERENCE TO RELATED APPLICATIONS

This application claims priority to U.S. Provisional Application 62/737,427, filed Sep. 27, 2018, the entirety of which is incorporated herein by reference.

### FIELD

The specification relates generally to inhalers, and more particularly to accessories for inhalers.

### BACKGROUND

Asthma and chronic obstructive pulmonary disease (COPD) are respiratory diseases which can be treated, in part, by administering medications via inhaler devices. A problem associated with poor asthma or COPD control is improper use of an inhaler. Improper inhaler technique can significantly affect the amount of medication reaching the lungs, hence patients with incorrect technique are likely to have poorly controlled asthma and COPD.

### SUMMARY

According to an aspect of the present disclosure, an accessory for an inhaler is provided. The accessory includes: a housing to couple to the inhaler; an accelerometer disposed in the housing, the accelerometer to obtain (i) motion data representing motion of the inhaler and (ii) orientation data representing an orientation of the inhaler; a microphone disposed in the housing, the microphone to obtain audio data for determining an inhalation rate of a user of the inhaler; a force sensor disposed in the housing, the force sensor to obtain force data representing force applied to the accessory; a communications interface disposed in the housing; a memory disposed in the housing; and a processor disposed in the housing, the processor interconnected with the communications interface and the memory, the processor to: detect actuation of the inhaler based on the force data applied to the accessory; and generate dosage administration data based on the motion data, the orientation data and the audio data.

According to an example implementation, the force sensor includes a spring and a tactile switch cooperating to detect inhaler actuation.

According to another aspect of the present disclosure, a method in an accessory for an inhaler is provided. The method includes: detecting actuation of the inhaler; obtaining audio data from a microphone of the accessory; generating flow data based on the audio data, the flow data representing a measured inhalation rate of a user of the inhaler; obtaining (i) motion data representing motion of the inhaler and (ii) orientation data representing an orientation of the inhaler; and generating dosage administration data based on the flow data, the motion data and the orientation data.

### BRIEF DESCRIPTION OF DRAWINGS

Implementations are described with reference to the following figures, in which:

FIG. 1 depicts a schematic diagram of an inhaler system including an accessory for an inhaler;

FIG. 2 is a block diagram of certain internal components of the accessory of FIG. 1;

FIG. 3 depicts sample flow rate comparison of a measured flow rate versus an estimated flow rate;

FIG. 4 depicts a flowchart of a method of operation of the accessory of FIG. 1;

FIG. 5 depicts a flowchart of a method of generating dosage administration data at block 430 of the method of FIG. 4; and

FIG. 6 depicts a system for managing and improving inhaler technique.

### DETAILED DESCRIPTION

A systemic review of errors in inhaler technique of metered dose inhalers showed that most errors were in coordination, speed, time, or depth of inspiration, and no post inhalation breath-hold. For dry powder inhalers and other breath-activated inhalers, there is a minimum inspiratory flow rate in order to activate the mechanism and deliver the medication to the user's lungs. Often, when a patient is struggling to breathe, they are not able to achieve the flow rate required to activate the breath-activated inhaler.

An accessory for inhalers is provided and may be implemented into various different types of inhalers, including metered dose inhalers, dry powder inhalers, and other breath-actuated inhalers. The accessory for metered dose inhalers includes a flow sensor, a motion sensor and a force sensor to detect actuation of the inhaler, and to record parameters including inhalation rate, breath-hold, orientation, shaking motion, and the like, around the time of actuation of the inhaler to provide feedback to the user on inhaler technique. This dosage administration data may be communicated to a client device, such as the user's phone. The data may additionally be pushed to a server (e.g. a cloud-based server) and used to provide specific recommendations, push notifications, and training to improve inhaler technique.

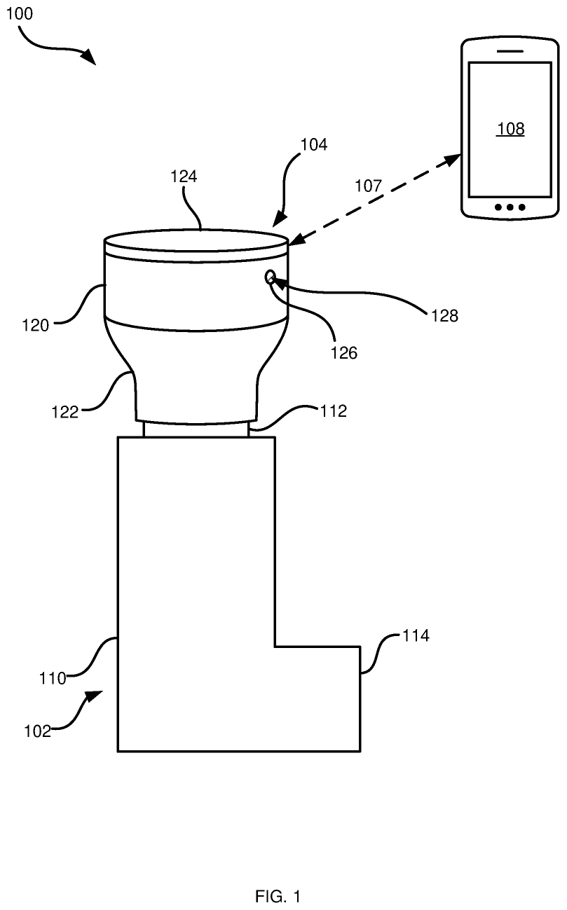

FIG. 1 depicts an system 100 including an inhaler 102 and an accessory 104.

The inhaler 102 includes a body 110 and a canister 112. The body 110 includes a receptacle to receive the canister 112 and a mouthpiece 114 to deliver an active ingredient to a user of the inhaler 102. In particular, the canister 112 is to contain the active ingredient of the inhaler 102 and is operatively coupled to the mouthpiece 114 to deliver the active ingredient to the user upon actuation of the inhaler 102. For example, the inhaler 102 may be actuated by depressing the canister 112 in the receptacle.

The accessory 104 includes a housing 120, which is generally configured to house internal components of the accessory 104 and to couple to the inhaler 102. In some examples, the housing 120 may couple to the body 110, while in other examples, the housing 120 may couple to the canister 112. The housing 120 may include metals, plastics (e.g. polyethylene terephthalate (PET)), 3D printed filament, combinations of the above, and the like.

The housing 120 may further include a seal element 122 to secure the accessory 104 to the inhaler 102. For example, the seal element 122 may be a flexible seal element, such as a frictional ring formed of rubbers, plastics, or the like, to fit onto the canister 112 via a friction fit. Further, the seal element 122 may be resilient to allow the accessory 104 to fit onto canisters of different sizes and/or shapes. In other examples, the seal element 122 may be a locking element and may cooperate with portions of the canister 112 to secure the accessory 104 to the canister. Generally, the seal element 122 secures the accessory 104 to the inhaler 102 such that the accessory 104 does not fall off upon movement or shaking of the inhaler 102.

The housing 120 may further include a cap 124 to enclose the internal components of the accessory 104. The cap 124 may further serve as a button to actuate the inhaler 102. In particular, the accessory 104 is secured to the inhaler 102 such that a user actuates the accessory 104 (as will be described further herein) simultaneously with actuating the inhaler 102. For example, in the present example, the user presses on the cap 124 of the accessory 104 to transfer force to the canister 112 to actuate the inhaler 102. Accordingly, the accessory 104 includes elements to detect the force on the cap 124 to infer actuation of the inhaler 102.

The inhaler system 100 may further include a client device 108 in communication with the accessory 104 via a wireless communication link 107. The client device 108 may be a mobile computing device such as a tablet, smart phone, or the like, or another computing device, such as a desktop computer, a laptop computer, a kiosk, or other suitable device.

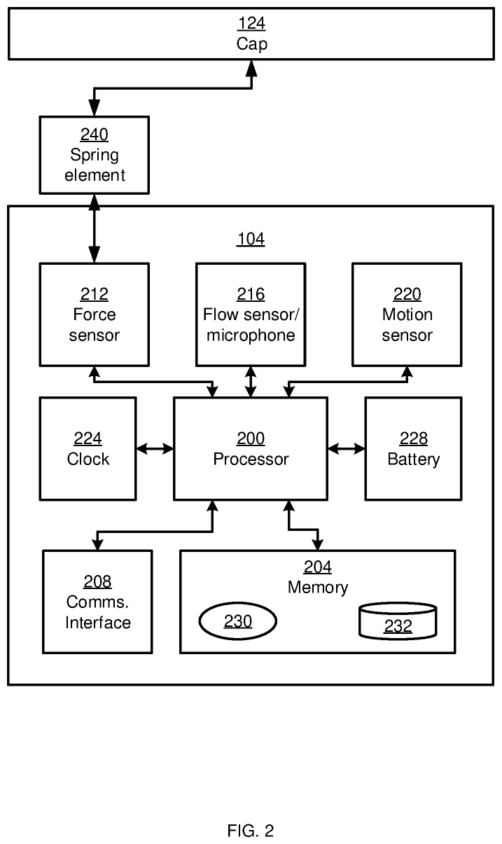

Referring now to FIG. 2, a block diagram of certain internal components of the accessory 104 is depicted. The internal components of the accessory 104 are generally disposed in the housing 120. For example, the internal components may be supported on a printed circuit board (PCB) or other suitable structures to support the components within the housing 120. The accessory 104 includes a processor 200, a non-transitory computer-readable storage medium, such as a memory 204, a communications interface 208, a force sensor 212, a flow sensor 216, and a motion sensor 220. The accessory 104 may further include a clock 224 and a battery 228.

The processor 200 may include a central-processing unit (CPU), a microcontroller, a microprocessor, a processing core, a field-programmable gate array (FPGA), or similar. The processor 200 may include multiple cooperating processors. The processor 200 may cooperate with the memory 204 to execute instructions to realize the functionality discussed herein. The memory 204 may include a combination of volatile (e.g. Random Access Memory or RAM) and non-volatile memory (e.g. read only memory or ROM, Electrically Erasable Programmable Read Only Memory or EEPROM, flash memory). All or some of the memory 204 may be integrated with the processor 200. The memory 204 stores applications, each including a plurality of computer-readable instructions executable by the processor 200. The execution of the instructions by the processor 200 configures the accessory 104 to perform various actions discussed herein. In particular, the applications stored in the memory 204 include a control application 230 to detect administration of a dosage from the inhaler and to analyze parameters of the dosage administration to generate dosage administration data. The memory 204 also includes a repository 232

The accessory 104 also includes the communications interface 208 interconnected with the processor 200. The communications interface 208 may configured for wireless communications (e.g. Bluetooth, other suitable short-range wireless communications protocols, Wi-Fi), and may include suitable hardware (e.g. transmitters, receivers, and the like) to allow the accessory 104 to communicate with other computing devices, such as the client device 108. The specific components of the communications interface 208 are selected based on the type of communication links 107 that the accessory 104 communicates over, as will be apparent to those of skill in the art.

The accessory 104 further includes the force sensor 212. The force sensor 212 is also interconnected with the processor 200 and is generally configured to capture force data representing force applied to the accessory 104. More particularly, the force sensor 212 may detect force applied to the cap 124. In particular, when the force detected by the force sensor 212 exceeds a threshold force, the processor 200 may determine that the inhaler 102 was actuated. In some examples, the accessory 104 may include a spring element 240 cooperating with cap 124, and the force sensor 212 may be a tactile switch. Specifically, tactile switch may capture force data representing a binary determination as to the activation of the tactile switch. The cap 124 and the tactile switch may cooperate such that the force to actuate the tactile switch corresponds to an actuation force to actuate the inhaler 102. In particular, the tension or resistance of the spring element 240 may be selected based on the actuation force to actuate the inhaler 102. That is, the force applied to the cap 124 and the spring element 240 to actuate the tactile switch is equal to the actuation force applied to the canister 112 to actuate the inhaler 102. Thus when the actuation force is supplied to the cap 124, the tactile switch is actuated, and may communicate to the processor 200 that the inhaler has been actuated.

The accessory 104 further includes the flow sensor 216 interconnected with the processor 200 and configured to capture flow data representing an inhalation rate of the user of the inhaler 102. In particular, the flow sensor 216 may be a microphone configured to capture audio data of the breathing (including inhalation, coughing, and the like) of the user. Accordingly, the flow sensor 216 may also be referred to herein as microphone 216. In other examples, other suitable flow sensors 216 may be utilized. In particular, suitable flow sensors may be configured to capture flow data representing inhalation rates of the user indirectly (i.e. not directly within the inhalation path between the canister 112 and the mouth of the user).

In the present example, the microphone 216 captures audio data (audio signal), and the processor 200 may correlate an estimated acoustic envelope from the audio signal to flow rate (i.e. inhalation rate). The acoustic envelope, calculated as the absolute value of the analytic signal, xₐ may computed based on equation (1):

xₐ=x+j\hat{x} (1)

In particular, x represents the audio signal and \hat{x} represents the Hilbert Transform of the audio signal. This value may be low-pass filtered with a cut-off frequency of 4 Hz to remove high frequency components of the signal. The analytical signal may be computed using a Hilbert transform. A power law regression law may be implemented to relate the estimated flow rate \hat{F} to the acoustic envelope xₑₙᵥ based on equation (2)

log \hat{F}=a*log xₑₙᵥ+b (2)

The variables a and b are calibration coefficients and may be determined via a calibration procedure (e.g. a least squares fitting) with a known flow rate measurement.

An experiment conducted in May 2019 showed that the audio signal is highly correlated to inhalation flow rate. The experiment consisted of measuring flow rate using a Venturi meter and gathering audio data from a USB microphone placed inside an enclosure. The inhaler was sealed inside the airtight enclosure to route all flow through the Venturi meter. A calibration was then performed using the above models to estimate flow rate and compare it to the measured values from the Venturi measurements.

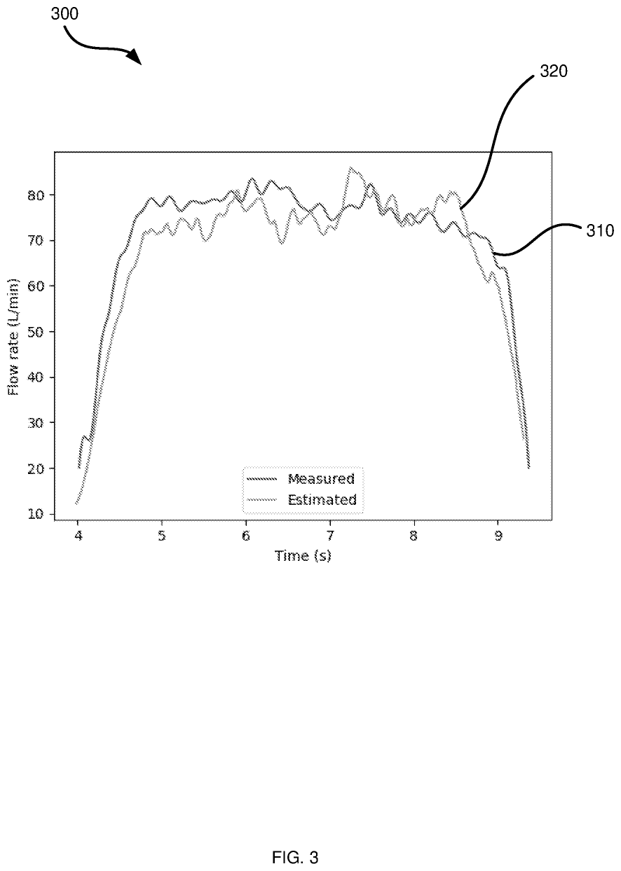

Fifteen samples taken were taken with peak flow rates varying between 60 L/min and 120 L/min. The calibration parameters a and b were calculated using Numpy library's polyfit function to apply a linear fit to the logarithmic signals of the measured and estimated flow rate. A sample flow rate comparison is depicted in FIG. 3. In particular, FIG. 3 depicts a plot 300 mapping a measured flow rate 310 and an estimated flow rate 320 against the flow rate (y-axis) and time (x-axis). In this example, the measured and estimated flow rate had an average error of 6.68%. On average, the results of the experiment showed that the audio estimate was highly correlated to the measured value, and could be matched within 7.25% accuracy on average.

The accessory 104 may further include an aperture 126 (depicted in FIG. 1) in the housing 120 to facilitate capturing the audio data by the microphone 216. The accessory 104 may further include a patch 128 overlaying the aperture 126 to reduce particulate matter entering the housing 120 while still allowing audio signals to be captured by the microphone 216.

The accessory 104 further includes the motion sensor 220, such as an accelerometer, gyroscope, or other suitable motion sensing element. The motion sensor 220 is interconnected with the processor 200 and is generally configured to capture orientation data and motion data of the accessory 104, and by extension, of the inhaler 102. In particular, the motion data may be transmitted to the processor 200 to identify specific motions of the inhaler 102, such as shaking (e.g. prior to use). In other examples, the motion sensor 220 may have an integrated circuit or other suitable processor capable of identifying the specific motions of the inhaler 102, and waking up the other components of the device, such as via a common interrupt function.

The clock 224 may be a real-time clock (e.g. in the form of an integrated circuit) to keep track of time for the device. In particular, the clock 224 may be interconnected with the processor 200 to allow tracking of the time data was obtained from the sensors 212, 216, and 220, including tracking the time of actuation of the inhaler. The battery 228 may be a lithium ion, lithium polymer, coin cell, AA, AAA battery, or the like, and may be disposable or rechargeable. Generally, the battery 228 is disposed in the housing and is used to power the processor 200 and the other components of the accessory 104.

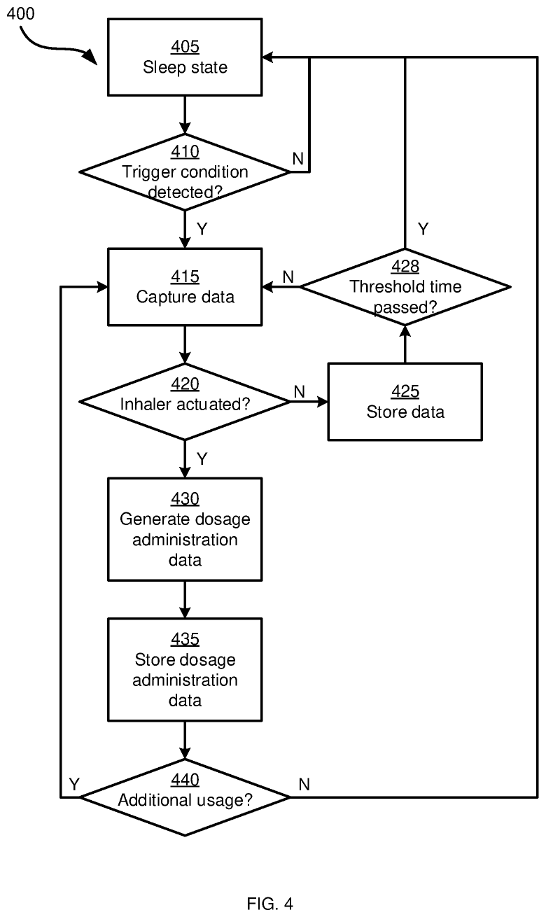

The functionality of the accessory 104, as implemented via execution of the control application 230 by the processor 200 will now be described in greater detail with reference to FIG. 4. FIG. 4 illustrates a method 400 of operation of an accessory for an inhaler, which will be described in conjunction with its performance in the system 100, and in particular, by the accessory 104, with reference to the components illustrated in FIGS. 1 and 2. In other examples, the method 400 may be performed by other suitable systems.

The method 400 begins at block 405. At block 405, the accessory 104 may be in a sleep state to conserve battery.

At block 410, it is determined whether a trigger condition is detected. The trigger condition may be, for example, detection of a “shaking” motion, as is described further below, based on motion data from the motion sensor 220. In other examples, the trigger condition may be detection of a certain orientation of the accessory 104 (e.g. indicative that the inhaler 102 is in an upright position in preparation for use) based on the orientation data from the motion sensor 220. That is, the determination may be made having regard to the orientation of the inhaler 102 being within a threshold angle (e.g. within 15°) of a predefined orientation (e.g. vertical). In further examples, the trigger condition may be other suitable motions, or a combination of motion data and orientation data (e.g. a near upright orientation combined with less erratic movement indicative of being manipulated by a user) which are indicative of preparation for use of the inhaler 102, and which are not triggered by other motions such as walking, running, or otherwise unintentional jostling of the inhaler 102 by the user.

If the determination at block 410 is negative, the method 400 returns to block 405, and the sleep state is maintained.

If the determination at block 410 is affirmative, the method 400 proceeds to block 415. At block 415, the components of the accessory 104 are awakened from the sleep state, for example, via a common interrupt function issued from the motion sensor. The force sensor 212, the flow sensor 216 and the motion sensor 220 capture, respectively, force data representing force applied to the accessory 104, flow data representing inhalation rates of the user, and motion data and orientation data of the accessory 104.

At block 420, the processor 200 determines whether the inhaler 102 has been actuated. In particular, the processor 200 may analyze the force data to determine whether the threshold force is detected.

If the determination at block 420 is negative, the method 400 proceeds to block 425. At block 425, the processor 200 stores the data captured at block 420 in the repository 232 in the memory 204. In particular, the processor 200 may further obtain a time stamp and may store the data in association with the time stamp. The accessory 104 may thus store data corresponding to periods prior to actuation of the inhaler 102, for future processing and/or analysis. The method 400 then proceeds to block 428.

At block 428, it is determined whether a threshold amount of time has passed. If the determination at block 428 is affirmative, it may be assumed that the motion sensor 220 detected a false positive, and the accessory 104 may return to the sleep state at block 405. If the determination at block 428 is negative, the method 400 returns to block 420 to continue capturing additional data.

If the determination at block 420 is affirmative, the method 400 proceeds to block 430. At block 430, the processor 200 obtains a time of actuation of the inhaler 102 from the clock 224 based on the time at which the force detected by the force sensor 212 exceeded the threshold force indicative of actuation of the inhaler 102. The processor 200 then generates dosage administration data for the actuation based on the flow data, the motion data, and the orientation data. In particular, the dosage administration data represents parameters affecting the effectiveness of the dosage administration.

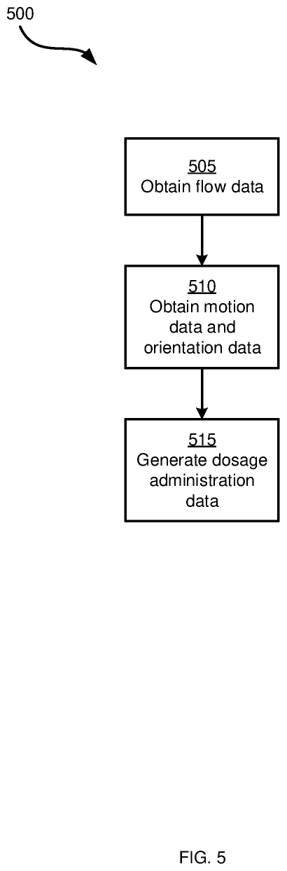

For example, referring to FIG. 5, a method 500 of generating dosage administration data is depicted. The method 500 will be described in conjunction with its performance in the system 100, and in particular by the accessory 104, with reference to the components illustrated in FIGS. 1 and 2. In other examples, the method 500 may be performed by other suitable systems.

The method 500 begins at block 505, for example, in response to detection of an actuation of the inhaler 102 by the processor 200. At block 505, the processor 200 obtains flow data. In particular, the processor 200 may obtain flow data for a first predetermined period prior to actuation, at the time of actuation, and for a second predetermined period after actuation. Accordingly, the processor 200 may obtain flow data corresponding to the first predetermined amount of time prior to actuation from the repository 232 in the memory 204. In particular, the processor 200 may obtain first audio data corresponding to the first predetermined period and may compute a first estimated flow rate based on the audio data. In other examples, the repository 232 may store a previously computed estimated flow rate, and hence, the processor 200 may retrieve the flow rate directly from the repository 232. The processor 200 may obtain flow data at the time of actuation, and for the second predetermined period from the flow sensor 216. In particular, the processor 200 may obtain actuation audio data from the microphone 216 at the time of actuation and may compute the estimated flow rate at the time of actuation based on the actuation audio data. The processor 200 may further obtain second audio data from the microphone 216 during the second predetermined period and may compute the second estimated flow rate during the second predetermined period based on the second audio data.

At block 510, the processor 200 obtains motion data and orientation data from the motion sensor 220. In particular, the processor 200 may obtain motion data and orientation data for a first predetermined period prior to actuation, at the time of actuation, and for a second predetermined period after actuation. In some examples, the periods prior to and after actuation during which motion data and orientation data are obtained may be the same as the periods during which flow data is obtained, while in other examples, the processor 200 may obtain motion data, orientation data, and flow data over different respective periods prior to and after actuation. Accordingly, the processor 200 may obtain the motion data and the orientation data corresponding to the period of time prior to actuation from the repository 232 in the memory. Further, the processor 200 may obtain motion data and orientation data at the time of actuation, and for the period after actuation from the motion sensor 220.

At block 515, the processor 200 generates dosage administration data based on the flow data, the motion data and the orientation data.

In particular, the inhalation rate of the user during inhalation is indicative of inhaler technique, and accordingly, is also representative of the effectiveness of the dosage administration. Specifically, the amount of the active ingredient which adheres to the inner walls of the body 110, or the user's tongue or back of the throat and not the user's lungs is directly proportional to the inhalation rate. Further, the audio data and subsequently computed inhalation rate may be used to measure lung function and provide data indicative of poor symptom control. In particular, generating the dosage administration may include storing the quantitative measured inhalation rate, comparing the measured inhalation rate to an ideal or average inhalation rate (e.g. expressed as a percentage or a ratio), determining whether the measured inhalation rate is within a threshold tolerance of the ideal or average inhalation rate, or similar. Additionally, the audio data and the subsequently computed inhalation rate may be used to determine proper breath holding of the user following actuation of the inhaler 102. In particular, proper breath holding ensures the medication reaches the lungs and is not released out of the lungs too soon. Accordingly, generating the dosage administration may include storing a measured breath-hold time, comparing the measured breath-hold time to an ideal or average breath-hold time (e.g. expressed as a percentage or a ratio), determining whether the measured breath-hold time is within a threshold tolerance of the ideal or average breath-hold time, or similar.

The motion data may be utilized to detect an appropriate “shaking” motion prior to actuation. In particular, inhalers are often to be shaken prior to use to disperse the active ingredient in the formulations, which may sink or rise in the canister. The processor 200 may detect one or more specific threshold values of the motion data to identify a “shaking” motion. In particular, the processor 200 may utilize vertical acceleration values as an indication of shaking. Further, the processor 200 may count a number of times that the vertical acceleration exceeds the threshold value and determine whether said threshold value is exceeded a threshold number of times. That is, the processor 200 may determine that the threshold vertical acceleration is to be exceeded at least twice to be identified as a shaking motion. In some examples, the processor 200 may be configured to stop counting and/or analyzing the motion data for shaking motion after the time of actuation of the inhaler 102, as the values for shaking are no longer relevant. The motion data may thus be utilized to identify sufficient “shaking” prior to actuation of the inhaler 102 and hence may inform the effectiveness of the dosage administration. Accordingly, generating the dosage administration data may include storing a binary indicator identifying detection of a shaking motion, storing a count of the shakes identified by the accessory 104, or similar.

The orientation data may be used to detect the orientation of the accessory 104, and by extension, the inhaler 102. In particular, the orientation of the inhaler 102 at the time of actuation affects the effectiveness of the medication reaching the user's lungs rather than the tongue or the roof of the mouth. The orientation data may thus be utilized to inform the effectiveness of the dosage administration. In particular, generating the dosage administration data may include storing a single value of the inhaler orientation at the time of actuation of the inhaler 102.

In some examples, generating the dosage administration data may further include aggregating the flow data, the motion data and the orientation data, for example, by a weighted sum or other predefined formula, to generate an overall dosage administration score.

Returning to FIG. 4, at block 435, the processor 200 stores the dosage administration data generated at block 430. For example, the processor 200 may store the dosage administration data in the repository 232 on the memory 204. In some examples, the dosage administration data and the associated time and date of actuation of the inhaler may additionally be communicated, via the communications interface 208, to the client device 108.

At block 440, the processor 200 determines whether additional usage of the inhaler 102 is detected after a predefined period of time (e.g. two minutes). Detection of additional usage may be based, for example, on the same or similar trigger conditions monitored at block 410. If no additional usage is detected at block 440, the method 400 proceeds to block 405, and the accessory 104 returns to a sleep state. If additional usage is detected, the method 400 proceeds to block 415 to capture further data.

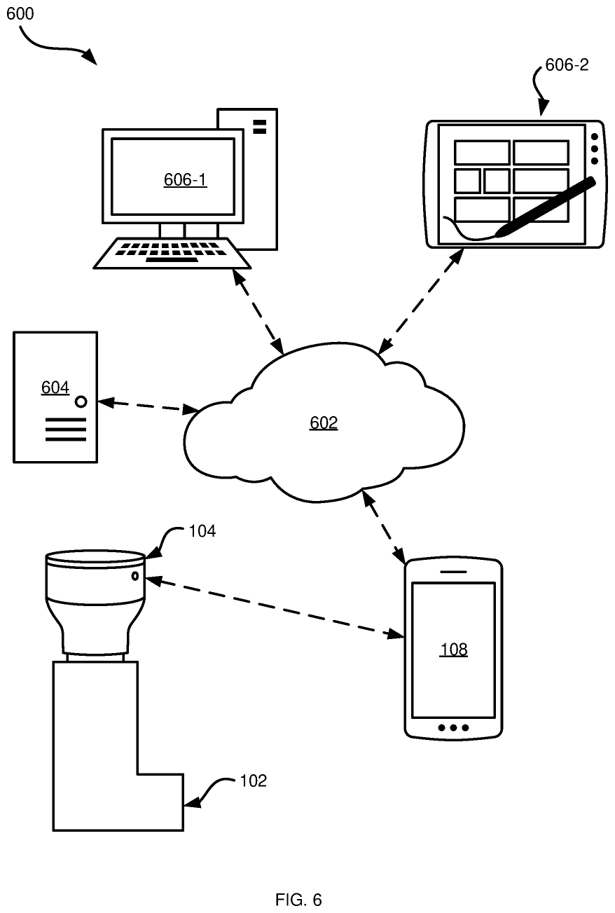

FIG. 6 depicts a system 600 for managing and improving inhaler technique. The system 600 includes the inhaler 102, the accessory 104, and the client device 108. The system 600 further includes a network 602, a server 604, and endpoint devices 606-1 and 606-2 (referred to herein generically as an endpoint device 606, and collectively as endpoint devices 606). The client device 108, the server 604 and the endpoint devices 606 are mutually coupled by the network 602 for data communications. Examples of suitable networks include internet protocol (IP) networks, such as intranet, a local-area network, a wide-area network, a virtual private network, a Wi-Fi network, a short-range wireless network, the internet, combinations of such, and similar.

The server 604 is generally configured to provide a platform for managing inhaler technique data for users. Specifically, the server 604 may store user accounts, including user identifiers, medical history and data, inhaler prescription data, inhaler usage data, including dosage administration data, and the like. The server 604 may further associate user accounts to authorized accounts to authorize access to certain user account data, such as to observe inhaler usage and dosage administration data. For example, authorized accounts may include physician accounts to allow physicians, hospitals, medical offices, and the like to access patient data. In other examples, authorized accounts may include parents and guardians, home-aid assistants, or other persons providing care to the primary user. The server 604 may further store technique data to provide feedback to the users based on the dosage administration data. In some examples, the server 604 may be implemented via one or more servers or computing devices as a cloud-based services. The server 604 may also store adherence data to provide feedback to the users based on the dosage administration data, and whether or not the users were adherent to their regularly prescribed medication plan.

The endpoint devices 606 are similar to the client device 108. In particular, the endpoint devices 606 may be mobile computing devices, such as tablets, smart phones, or the like, or other computing devices, such as desktop computers, laptop computers, kiosks, or other suitable devices. Generally, the endpoint devices 606 may be utilized by authorized physicians, hospitals, medical offices, and the like to connect to the server 604 via the network 602 to access the patient data. In some examples, the endpoint devices 606 may also be utilized by authorized parties, such as parents or guardians, caretakers, and the like.

In operation, the accessory 104 transmits the dosage administration data via the wireless communication link 107 to the client device 108. The client device 108, in turn, may relay the dosage administration data to the server 604 via the network 602. In particular, the client device 108 may further associate account data, such as a user identifier, with the dosage administration data prior to sending the dosage administration data to the server 604. Authorized users at the endpoint devices 606 may then access the dosage administration data associated with the user account and may provide feedback. For example, a physician may provide a specific suggestion for improving inhaler technique based on the dosage administration data (e.g. to begin inhalation sooner, or the like) and may communicate the suggestion via the platform provided by the server 604. Accordingly, the feedback provided by the physician may be transmitted to the client device 108 for the user. In other examples, feedback may be automatically generated by the server 604, for example, based on an algorithmic analysis of the dosage administration data.

The client device 108 may further be configured, via a control application stored at the client device 108, to display a dashboard containing user account data, including dosage administration data. For example, the dashboard may include prescribed inhaler data, historical data regarding prior dosage administrations, current dosage administration data, graphical representations of the various data, and the like. In some examples, the dashboard may further provide feedback and recommendations based on the dosage administration data. For example, the dashboard may provide feedback from the physician, or based on the automatically generated feedback from the server 604. In some examples, the dashboard may further display reminders, for example to remind users to use the inhaler in accordance with the prescribed inhaler data. In particular, the control application may provide push notifications on a mobile device.

In other examples, other implementations and variations are contemplated. For example, some or all of the analysis of the dosage administration data may be performed at the client device 108, at the server 604, or both. In still further examples, the accessory 104 may provide the raw data captured by the sensors to the client device 108, and the dosage administration data may be generated by the client device 108, the server 604, or both.

Thus, the present disclosure provides an accessory for an inhaler to record parameters before, during, and after dosage administration from the inhaler. The parameters are analyzed to generate dosage administration data indicative of the effectiveness of the dosage administration to administer medication to the user. Further, the present disclosure provides a platform for managing inhaler technique based on the dosage administration data. In particular, dosage administration data may be accessed by medically interested parties, including physicians, and feedback may be provided to the user to improve inhaler technique.

The scope of the claims should not be limited by the embodiments set forth in the above examples, but should be given the broadest interpretation consistent with the description as a whole.

1. An accessory for an inhaler, the accessory comprising:
a housing to couple to the inhaler;
an accelerometer disposed in the housing, the accelerometer to obtain (i) motion data representing motion of the inhaler and (ii) orientation data representing an orientation of the inhaler;
a microphone disposed in the housing, the microphone to obtain audio data for determining an inhalation rate of a user of the inhaler;
a force sensor disposed in the housing, the force sensor to obtain force data representing force applied to the accessory;
a communications interface disposed in the housing;
a memory disposed in the housing; and
a processor disposed in the housing, the processor interconnected with the communications interface and the memory, the processor to:
detect actuation of the inhaler based on the force data applied to the accessory; and
generate dosage administration data based on the motion data, the orientation data and the audio data.

2. The accessory of claim 1, further comprising a seal element coupled to the housing, the seal element to secure the accessory to the inhaler.

3. The accessory of claim 2, wherein the seal element comprises a flexible seal element.

4. The accessory of claim 1, wherein the housing further comprises an aperture to facilitate capturing the audio data by the microphone.

5. The accessory of claim 4, further comprising a patch overlaying the aperture, the patch to reduce particulate matter entering the housing.

6. The accessory of claim 1, wherein the force sensor comprises a tactile switch; and wherein the force data comprises a binary determination as to an activation of the tactile switch.

7. The accessory of claim 6, further comprising a spring element and a cap; and wherein the spring element and the cap are configured to cooperate with the tactile switch to detect actuation of the inhaler.

8. The accessory of claim 7, wherein the spring element is selected based on an actuation force to actuate the inhaler.

9. A method in an accessory for an inhaler, the method comprising:
detecting actuation of the inhaler;
obtaining audio data from a microphone of the accessory;
generating flow data based on the audio data, the flow data representing a measured inhalation rate of a user of the inhaler;
obtaining (i) motion data representing motion of the inhaler and (ii) orientation data representing an orientation of the inhaler; and
generating dosage administration data based on the flow data, the motion data and the orientation data.

10. The method of claim 9, wherein detecting actuation of the inhaler comprises:
obtaining force data from a force sensor of the accessory, the force data representing a force applied to the accessory; and
when the force data exceeds a threshold force, identifying the actuation of the inhaler.

11. The method of claim 9, further comprising determining a time of actuation of the inhaler.

12. The method of claim 11, wherein obtaining the audio data comprises:
obtaining first audio data for a first predetermined period prior to the time of actuation of the inhaler;
obtaining actuation audio data at the time of actuation of the inhaler; and
obtaining second audio data for a second predetermined period after the time of actuation of the inhaler.

13. The method of claim 11, wherein obtaining the motion data and the orientation data comprises:
obtaining first motion data and first orientation data for a first predetermined period prior to the time of actuation of the inhaler; and
obtaining second motion data and second orientation data for a second predetermined period after the time of actuation of the inhaler.

14. The method of claim 9, wherein generating dosage administration data comprises one or more of:
storing the measured inhalation rate;
comparing the measured inhalation rate to an ideal inhalation rate;
determining whether the measured inhalation rate is within a threshold tolerance of the ideal inhalation rate;
storing a measured breath-hold time;
comparing the measured breath-hold time to an ideal breath-hold time;
determining whether the measured breath-hold time is within a threshold tolerance of the ideal breath-hold time;
storing a binary indicator identifying detection of a shaking motion;
storing a count of shakes identified;
storing a single value of inhaler orientation at a time of actuation of the inhaler; and
aggregating the flow data, the motion data, and the orientation data to generate an overall dosage administration score.

15. The method of claim 9, further comprising storing the dosage administration data in a memory of the accessory.

16. The method of claim 9, further comprising communicating the dosage administration data to a client device via a wireless communication link.

17. The method of claim 9, further comprising, prior to detecting the actuation, waking the accessory from a sleep state in response to one or more of:
detecting a shaking motion; and
detecting the orientation of the inhaler being within a threshold angle of a predefined orientation.

18. The method of claim 17, wherein detecting the shaking motion comprises detecting a vertical acceleration exceeding a threshold value.

19. The method of claim 18, wherein detecting the shaking motion further comprises determining whether the threshold value is exceeded a threshold number of times.
